(19) United States
(12) Patent Application Publication
(10) Pub. No.: US 2023/0119686 A1
(43) Pub. Date: Apr. 20, 2023

(54) METHOD FOR IMPROVING SKIN CONDITION WITH REDLOVE APPLES EXTRACT

(71) Applicant: TCI CO., LTD., Taipei (TW)

(72) Inventors: YUNG-HSIANG LIN, Taipei (TW); MARGARET LAI, Taipei (TW)

(21) Appl. No.: 17/816,431

(22) Filed: Aug. 1, 2022

Related U.S. Application Data
(60) Provisional application No. 63/228,169, filed on Aug. 2, 2021.

(51) Int. Cl. A61K 8/9789; A61Q 19/00

(52) U.S. Cl. CPC A61K 8/9789; A61Q 19/00

(57) ABSTRACT

Method for improving skin condition with Redlove apples extract includes administering to a subject in need thereof a composition including an effective dose of the Redlove apples extract, wherein the Redlove apples extract was prepared by extracting Redlove apples (Malus pumila) with water at 80° C. to 90° C. for 45 min to 75 min.

### CROSS-REFERENCE TO RELATED APPLICATION

This application claims the benefit of U.S. provisional application Ser. No. 63/228,169, filed on Aug. 2, 2021. The entirety of the above-mentioned patent application is hereby incorporated by reference herein and made a part of the specification.

### REFERENCE OF AN ELECTRONIC SEQUENCE LISTING

The contents of the electronic sequence listing (P212305US1_ST26.xml; Size: 8 KB; and Date of Creation: Jul. 28, 2022) is herein incorporated by reference in its entirety.

### BACKGROUND

### Technical Field

The instant disclosure relates to a method for improving skin condition, and particularly relates to a method for improving skin condition with a Redlove apples extract prepared from Redlove apples (Malus pumila).

### Related Art

Along with the changes over time, people pursue perfect appearance and have high requirements from appearance outline, skin quality, skin elasticity, skin texture and posture to internal collagen content. Healthy skin and skin color are a major factor for maintaining a beautiful appearance, so people pay more and more attention to the health and maintenance of skin and keep the skin in the best state from inside to outside.

In order to solve the above problems, it is urgent for those skilled in the art to develop functional foods to solve the problems and benefit vast population with such requirements.

### SUMMARY

In view of this, the instant disclosure provides a Redlove apples extract prepared from Redlove apples (Malus pumila), which has a function of improving skin condition.

In some embodiments, application of the Redlove apples extract in preparation of a composition for improving the skin condition is provided, and the Redlove apples extract is prepared by extracting Redlove apples (Malus pumila) with water at 95° C. for 1 h.

In some embodiments, a method for improving skin condition with the Redlove apples extract is provided, which includes the step of administering to a subject in need thereof a composition including an effective dose of the Redlove apples extract, and the Redlove apples extract is prepared by extracting Redlove apples (Malus pumila) with water at 80° C. to 90° C. for 45 min to 75 min.

In some embodiments, improving the skin condition includes reducing wrinkles, reducing skin textures, making the skin ruddy and glossy, or a combination thereof.

In some embodiments, the Redlove apples extract contains at least 129.1 ppm of chlorogenic acid.

In some embodiments, the Redlove apples extract has a capability of increasing an expression level of elastin synthesis related gene.

In some embodiments, the elastin synthesis related gene includes FAN gene.

In some embodiments, the Redlove apples extract has a capability of increasing skin elastin content.

In some embodiments, the Redlove apples extract has a capability of increasing an expression level of skin moisturizing related genes.

In some embodiments, the skin moisturizing related genes include Keratin 14 gene, GBA gene and HAS gene.

In some embodiments, the dosage of the composition is at least 0.6 g/day of the Redlove apples extract in liquid form.

In some embodiments, the effective dose of the Redlove apples extract in liquid form is at least 0.6 g/day.

In summary, the Redlove apples extract in any one of the embodiments is prepared by extracting the Redlove apples (Malus pumila) with water at 95° C. for 1 h, and can be used for improving skin condition. In some embodiments, improving skin condition includes reducing wrinkles, reducing skin textures, making the skin ruddy and glossy, or a combination thereof. In some embodiments, the Redlove apples extract has the functions of increasing the expression level of elastin synthesis related gene (such as FBN1 gene), increasing skin elastin content, increasing the expression level of skin moisturizing related genes (such as Keratin 14 gene, GBA gene and HAS gene) and the like, so that the skin condition can be effectively improved (such as the functions of reducing the wrinkles, reducing the skin textures, and making the skin ruddy and glossy).

### BRIEF DESCRIPTION OF THE DRAWINGS

FIG. 1 is a data analysis diagram of content of chlorogenic acid in the Redlove apples extract;

FIG. 2 is a data analysis diagram of relative expression level of elastin synthesis related gene;

FIG. 3 is a data analysis diagram of relative secretion of elastin;

FIG. 4 is a data analysis diagram of relative expression level of skin moisturizing related gene;

FIG. 5 is a data analysis diagram of average detection quantity of wrinkles of a subject at the 0^{th} week and the 4^{th} week;

FIG. 6 is an analysis photograph of wrinkles of one subject at the 0^{th} week and the 4^{th} week;

FIG. 7 is a data analysis diagram of average detection quantity of skin textures of a subject at the 0^{th} week and the 4^{th} week;

FIG. 8 is an analysis photograph of skin textures of one subject at the 0^{th} week and the 4^{th} week; and

FIG. 9 is a photograph of skin conditions of two subjects at the 0^{th} week and the 4^{th} week.

### DETAILED DESCRIPTION

Some specific implementations of the instant disclosure is described below. The instant disclosure may still be practiced in many different forms without departing from the spirit of the instant disclosure, and the scope of protection should not be limited to the conditions specifically stated in this specification.

Statistical analysis is performed by Excel software in the instant disclosure. Data is represented by mean value±standard deviation (SD), and a difference between groups is analyzed by student's t-test. In the figures, “*” represents that the p value is less than 0.05, “**” represents that the p value is less than 0.01, and “***” represents that the p value is less than 0.001. The more “*”, the more significant the statistical difference.

Numerical values of experimental data used in the instant disclosure are approximate values, and all experimental data is represented within a range of ±10%, preferably within a range of ±5%.

In the instant disclosure. “wt %” represents weight percentage, and “vol %” represents volume percentage.

The term “extract” refers to a product prepared by extraction. The extract can be presented in a form of a solution dissolved in a solvent, or the extract can be presented in a form of a concentrate or essence which contains no or substantially contains no solvent, and can also be presented in a form of powder after drying.

The Redlove apples are scientifically named as Malus pumila, commonly known as red-fleshed apples, and are well known by red flesh, red peel and red flowers, and are mainly produced in Australia, such as Australian Lenswood farm. The Redlove apples are obtained by inoculating pollen of a red-fleshed plant and an scab-resistance plant, therefore they are not a new variety subjected to genetic modification. Moreover, the Redlove apples can be prevented from discoloring after being contacted with air for a long time, they can be kept bright red after being cooked or juiced, and their nutritional value is higher than common apples. In some embodiments, the synonyms of the Redlove apples include Malus niedzwetzkyana Dieck, Malus pumila var. niedzwetzkyana Schneid, Malus domestica ‘Redlove Group’ and the like. In some embodiments, the variety of the Redlove apples is LUBA11706.

In some embodiments, the Redlove apples extract is prepared by crushing and filtering red-fleshed fruits of the Redlove apples (Malus pumila), mixing the crushed and filtered red-fleshed fruits with an extraction solvent according to a certain weight ratio, performing extraction at a specific temperature to obtain a primary extract liquid containing solids, filtering the primary extract liquid to remove fine solid impurities, concentrating the filtered primary extract liquid to obtain a concentrated liquid, and sterilizing the concentrated liquid to obtain a sterilized Redlove apples extract in liquid form, or drying the concentrated liquid into powder in a spray drying manner to obtain a Redlove apples extract in solid form. Therefore, the extraction efficiency can be significantly improved by a specific ratio of the extraction solvent and a to-be-extracted substance (such as crushed fruits) or specific extraction time; and moreover, due to the specific extraction time, possible degradation of active ingredients in the extract caused by overlong extraction time can be avoided.

In some embodiments, the Redlove apples extract is prepared by crushing red-fleshed fruits of the Redlove apples (Malus pumila), and filtering to remove overlarge particles to obtain Redlove apples powder; and then, mixing water and the Redlove apples powder at a weight ratio of 10-30:1-3, and extracting at 80° C. to 90° C. for 45 min to 75 min. For example, the Redlove apples extract is prepared by mixing the crushed fruit powder of the Redlove apples and water at a weight ratio of 1:20, and extracting at 85° C.±5° C. for about 60 min.

In some embodiments, the Redlove apples powder is prepared by crushing the Redlove apples, sieving the crushed Redlove apples by a 30-mesh screen and removing overlarge particles.

In some embodiments, the Redlove apples powder and water are mixed at a weight ratio of 1-3:10-30 and are extracted at a temperature of 80° C. to 90° C. for 45 min to 75 min to obtain a primary extract liquid, and the primary extract liquid is filtered by a 400-mesh filtering screen to remove fine solids. The filtered primary extract liquid is subjected to reduced pressure concentration at a temperature of 50° C.-82° C. until the Degrees Brix of the primary extract liquid is 12.0±0.5, so as to obtain the Redlove apples extract. For example, the temperature of the reduced pressure concentration can be 60° C.±5° C.

In some embodiments, the fruits of the Redlove apples include peels, pulp and seeds.

In some embodiments, the Redlove apples extract contains at least 129.1 ppm of chlorogenic acid. Moreover, compared with other apple varieties, the Redlove apples (Malus pumila) has higher chlorogenic acid content than that of other varieties of white-fleshed apples (such as Malus domestica Borkh.).

In some embodiments, the Redlove apples extract has the function of improving skin condition. For example, improving the skin condition includes reducing wrinkles, reducing skin textures, making the skin ruddy and glossy, or a combination thereof. In other words, after a subject takes the Redlove apples extract, the wrinkles and the skin texture can be effectively reduced, and/or the skin is ruddy and glossy.

In some embodiments, the Redlove apples extract has the capability of increasing an expression level of an elastin synthesis related gene. For example, the elastin synthesis related gene includes an FBN1 gene. Proteins encoded by the FBN1 gene are main structures of microfibers, and the microfibers mainly provide connection between zonule, elastin and other connective tissues. In other words, after a subject takes the Redlove apples extract, the skin of the subject keep elastic like a spring. In one embodiment, the expression level of the FBN1 gene is effectively increased by 1.7 fold(s) by the Redlove apples extract.

In some embodiments, the Redlove apples extract has the capability of increasing skin elastin content. Specifically, elastin is synthesized by skin fibroblast, it is one of important elements forming skin elastin fibers and is closely related to the elasticity of the skin. In other words, after a subject takes the Redlove apples extract, the elastin content can be increased, then the elasticity of the skin is improved, skin collapse and wrinkle are reduced, and as a result, skin aging is delayed. In one embodiment, the skin elastin content is effectively increased by 1.93 fold(s) by the Redlove apples extract.

In some embodiments, the Redlove apples extract has the capability of improving an expression level of skin moisturizing gene. For example, elastin synthesis related genes include Keratin 14 gene, GBA gene and HAS gene. Specifically, the Keratin (Krt) gene is capable of regulating and controlling the synthesis of keratin on an outer layer of the skin and is a main component of the skin. GBA protein encoded by the Glucocerebrosidase (GBA) gene is an enzyme for synthesizing ceramide, so the skin can be regulated and controlled to synthesize ceramide to supplement cell gaps of the stratum corneum of the skin and enhance the water locking capability of the skin. The Hyaluronan synthase (HAS) gene can participate in the synthesis of hyaluronic acid with different molecular weights of the skin. In other words, after a subject takes the Redlove apples extract, the expression of the keratinocyte moisturizing related gene such as the Keratin (Krt) gene (e.g., Keratin 14 gene), the GBA gene and the HAS gene (e.g., HAS2 gene) can be effectively improved, thus the skin structure is enhanced, the cell gaps are reduced, the optimal moisturizing factor is provided for the skin, and the skin is softer and well-moisturized.

In some embodiments, the Redlove apples extract can effectively smooth fine lines, reduce the roughness of the skin and make the skin ruddy and glossy or achieve a combination thereof. Specifically, the Redlove apples extract can reduce skin wrinkles, smooth fine lines, reduce skin textures, reduce the roughness of the skin and/or make the skin ruddy and glossy, so as to improve the skin condition.

In some embodiments, the subject is a human.

In some embodiments, any one of the abovementioned compositions can be a pharmaceutical product. In other words, the pharmaceutical product includes an effective content of the Redlove apples extract.

In some embodiments, the pharmaceutical product can be prepared into a dosage form suitable for being administrated through the intestinal tract, or administrated parenterally, orally or topically by utilizing a technology specifically known by the skilled.

In some embodiments, the dosage form for intestinal administration or oral administration can be, but not limited to, tablet, troche, lozenge, pill, capsule, dispersible powder or granule, solution, suspension, emulsion, syrup, elixir, slurry or the like. In some embodiments, the dosage form suitable for being administrated parenterally or topically can be, but not limited to, injection, sterile powder, external preparation or the like. In some embodiments, the administration manner of the injection can be subcutaneous injection, intraepidermal injection, intradermal injection or intralesional injection.

In some embodiments, the pharmaceutical product can include a pharmaceutically acceptable carrier widely used for pharmaceutical manufacturing technologies. In some embodiments, the pharmaceutically acceptable carrier can be one or more of the following carriers: solvent, buffer, emulsifier, suspending agent, decomposer, disintegrating agent, dispersing agent, binding agent, excipient, stabilizing agent, chelating agent, diluent, gelling agent, preservative, wetting agent, lubricant, absorption delaying agent, liposome and the like. Selecting the types and the quantity of the carriers is within the scope of professional quality and routine techniques of people who are familiar with the technology. In some embodiments, the solvent serving as the pharmaceutically acceptable carrier can be water, normal saline, phosphate buffered saline (PBS) or aqueous solution containing alcohol.

In some embodiments, any one of the abovementioned compositions can be an edible product. In other words, the edible product includes a specific content of the Redlove apples extract. In some embodiments, the edible product can be a general food, a healthcare food or a dietary supplement.

In some embodiments, the edible product can be prepared into a dosage form suitable for oral administration by utilizing a technology specifically known by the skilled. In some embodiments, the general food can be the edible product itself. In some embodiments, the general food can be but not limited to beverages, fermented foods, bakery products or seasonings.

In some embodiments, the obtained Redlove apples extract can be further used as a food additive to prepare a food composition including the Redlove apples extract. Therefore, the Redlove apples extract of any embodiment can be added during raw material preparation by a known method, or the Redlove apples extract of any embodiment is added in the food preparation process to prepare an edible product (namely the food composition) with any edible material for human beings and non-human animals to eat.

In some embodiments, in the composition including the Redlove apples extract, the Redlove apples extract included can be in a liquid form or a solid form. For example, the Redlove apples extract in the solid form can be powder or a lozenge.

In some embodiments, the dosage of the composition is at least 0.6 g/day of the Redlove apples extract in liquid form. That is, the effective dose of the liquid Redlove apples extract can be at least 0.6 g/day.

In some embodiments, the dosage of the composition is at least 0.3 g/day of the Redlove apples extract in solid form. That is, the effective dose of the solid Redlove apples extract can be at least 0.3 g/day.

### Embodiment 1: Preparation of Redlove Apples Extract

Firstly, red-fleshed fruits (produced in Australian Lenswood farm) of Redlove apples (Malus pumila) were prepared and crushed by a 30-speed blender (Brand: Osterizer). Then, the crushed Redlove apples fruits were sieved by a 30-mesh screen to remove overlarge particles, so as to obtain Redlove apples powder. Then, water and the Redlove apples powder were mixed at a weight ratio of 20:1, and the mixture was extracted at a temperature of 85±5° C. for 60 min to form primary extract liquid containing solids.

Then, the primary extract liquid containing solids was filtered by a 400-mesh filtering screen to remove fine solids in the primary extract liquid. Finally, the filtered primary extract liquid was subjected to reduced pressure concentration at a temperature of 60° C.±5° C. by a concentrator (Brand/Model: BUCHI—Rotavapor R-100) until the Degrees Brix of the solution was 12.0±0.5, so as to obtain a liquid Redlove apples extract.

### Embodiment 2: Analysis of Chlorogenic Acid Content

Quantitative analysis was performed on chlorogenic acid of die liquid Redlove apples extract prepared in Embodiment 1 and apple juice of Malus domestica Borkh (purchased from AGRANA Juice; 100% apple juice). The liquid Redlove apples extract was treated as an experimental group, and the apple juice was treated as a control group and also represented an extract of white-fleshed apples (Malus domestica Borkh).

Firstly, the liquid Redlove apples extract prepared in Embodiment 1 and apple juice were filtered by a 0.22 μm PVDF filter membrane (Polyvinylidene fluoride membrane filters, PVDF, Millipore, USA), and 10 μl of the filtered Redlove apples extract and the commercially available apple juice were respectively taken as samples of the experimental group and the control group.

Chlorogenic acid solutions with concentrations of 10 μg/mL, 20 μg/mL, 50 μg/mL, 80 μg/mL, and 100 μg/m were respectively prepared from chlorogenic acid (Brand: Merck, Taiwan) and deionized water to serve as chlorogenic acid standard solutions.

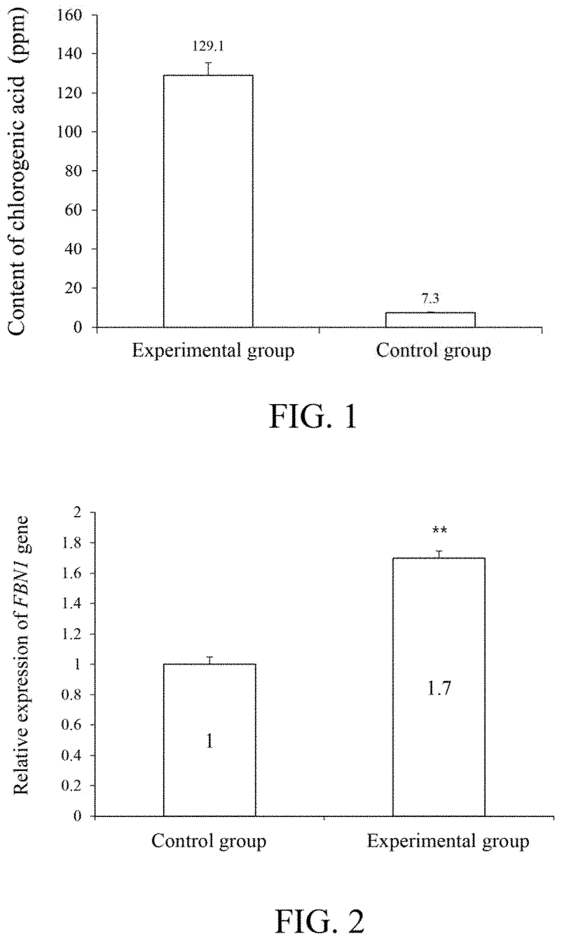

Then, the content of the chlorogenic acid in the two groups was analyzed by a high performance liquid chromatography (HPLC, Hitachi, Tokyo, Japan). 10 μl of the sample in the experimental group and the chlorogenic acid standard solutions with different concentrations in the abovementioned 5 groups were respectively taken and injected into the high performance liquid chromatography for liquid chromatography, and the retention time of wave crests obtained by the sample in the experimental group and the chlorogenic acid standard solutions and an absorption spectrum were compared. After drawing a calibration curve with UV wavelength absorbance values in linear positive correlation obtained by detecting the chlorogenic acid standard solutions with different concentrations in the 5 groups, quantitative analysis was performed on the content of the chlorogenic acid in the experimental group. The experimental steps of the control group were shown as the above, and 10 μl of the sample in the control group and the chlorogenic acid standard solutions with different concentrations in the abovementioned 5 groups were respectively taken and analyzed by the high performance liquid chromatography, and the content of the chlorogenic acid in the control group was calculated. The analysis result was shown in FIG. 1.

The high performance liquid chromatography belonged to Hitachi chromaster 5260 series; a used HPLC analysis tubular column was Mightysil RP-18 GP 250 (250×4.6 mm, 5 μm, Kanto, Tokyo, Japan); a used elution solvent was conveyed by Hitachi chromaster 5110; a used tubular column constant temperature device was Hitachi chromaster 5310; a used diode array detector (DAD) was Hitachi chromaster 5430; and a used detection wavelength was 190 nm-800 nm.

Moreover, the set elution conditions were as follows: the flow rate was 1 mL/min, the column temperature was 40° C., and the sample injection amount was 10 μl. The volume ratio of a solution A to a solution B was 2:98 at 0 min, the volume ratio of the solution A to the solution B was 2:98 at 10 min, the volume ratio of the solution A to the solution B was 70:30 at 40 min, the volume ratio of the solution A to the solution B was 100:0 at 50 min, and the volume ratio of the solution A to the solution B was 100:0 at 60 min.

The used solution A was methanol (HPLC grade; purchased from Merck, Taiwan) and 0.1% formica acid (ACS grade; purchased from Merck, Taiwan) was additionally added. The used solution B was water and 0.1% formica acid was additionally added, and the water was ultrapure water (18.2 MCI) from a Millipore Synergy® water preparation system (Millipore, USA).

As shown in FIG. 1, the content of chlorogenic acid of the control group was 7.3 ppm, and the content of chlorogenic acid of the experimental group was 129.1 ppm. That is, the content of chlorogenic acid in the Redlove apples extract was at least 17 fold(s) that of the commercially available apple juice, which means that the content of chlorogenic acid in the Redlove apples extract was significantly higher than that of the general white-fleshed apples extract.

### Embodiment 3: Analysis Experiment of Elastin Synthesis Related Gene

Culture medium used was an Eagle's minimum essential medium (hereinafter referred to as MEM culture medium) containing 10 vol % of fetal bovine serum (FBS; Brand: Gibco), 1 mM of sodium pyruvate (90%; Brand: Gibco), 0.1 mM of Non-Essential Amino Acids (Brand: Gibco) and 1.5 g/L of sodium bicarbonate (Brand: Gibco). Cell strain used was human skin fibroblast (CCD-966Sk cell, Brand: ATCC®, CRL-1881), hereinafter referred to as CCD-966Sk cells. The analyzed elastin synthesis related gene was Fibrillin-1 (FBN1) gene (GeneID: 2200).

The CCD-996SK cells were seeded in a 6-well culture dish containing 2 ml of MEM culture medium per well at an amount of 1.5×10⁵ cells per well, cultured at 37° C. for 24 h, and divided into an experimental group and a control group. Then, the MEM culture medium was replaced with an experimental culture medium, and cultured for 24 h. The experimental culture medium in the experimental group was an MEM culture medium containing 0.125 mg/mL of Redlove apples extract prepared in Embodiment 1. The experimental culture medium in the control group was a pure MEM culture medium (namely the MEM culture medium without the Redlove apples extract).

Then, RNAs of CCD-996SK cells cultured by the experimental culture medium of each group were extracted by an RNA extraction kit (Brand: Genemark). 1000 ng of extracted RNA was taken from each group to serve as a template, and the RNA from each group was translated into cDNA by a cDNA synthesis reagent (purchased from Geneaid Company, Taiwan) and SuperScript® III reverse transcriptase. Then, quantitative real-time reverse transcription polymerase chain reaction was performed by a KAPA CYBR FAST qPCR kit (KAPA Biosystems) and an AB Step One Plus™ Real-Time PCR system in combination with an FBN1-F (SEQ ID NO: 1) primer and an FBN1-R (SEQ ID NO: 2) primer (shown in Table 1), so as to perform quantitative analysis on a target gene, and the analysis result was shown in FIG. 2. The instrument setting conditions of the quantitative real-time reverse transcription polymerase chain reaction were that: the reaction was performed at 95° C. for 23 s and performed at 60° C. for 30 s, and 40 cycles in total; and the cDNA PCR size of the FBN1 gene was 166 bp. Particularly, the gene expression in FIG. 2 was relatively presented, the standard deviation was calculated by an STDEV formula of Excel software, and the statistical significant difference among the groups was statistically analyzed by a student's t-test. In FIG. 2, “**” represented that the p value was less than 0.01 under the comparison with the control group.

TABLE 1

| Sequence | Sequence | | |
|---|---|---|---|
| number | name | Sequence (5′→3′) | Length |
| SEQ ID NO: 1 | FBN1-F | TTTAGCGTCCTACACGAGCC | 20 |
| SEQ ID NO: 2 | FBN1-R | CCATCCAGGGCAACAGTAAGC | 21 |

As shown in FIG. 2, the expression level of the FBN1 gene in the control group was regarded as 1, which means that the expression level of the FBN1 gene in CCD-996SK cells without being subjected to treatment of the Redlove apples extract was regarded as 100%. Therefore, the expression level of the FBN1 gene in the experimental group was 1.7. In other words, the expression level of the FBN1 V gene in CCD-996SK cells subjected to treatment of the Redlove apples extract was improved by 1.7 fold(s).

Therefore, the Redlove apples extract had an effect of improving the expression level of the FBN1 gene. In other words, after a subject taken the Redlove apples extract, the expression level of the FBN1 gene in the subject was improved, and then FBN1 protein was generated; and the FBN1 protein serving as one of main structures of microfibers keep the elasticity of the skin like a spring.

### Embodiment 4: Detection of Elastin Content

Herein, cell culture medium (hereinafter referred to as MEM culture medium) containing 90 vol % of minimum essential medium (Brand: Gibco), 10 vol % of fetal bovine serum (FBS: Brand: Gibco) and 1 mM of sodium pyruvate (Brand: Gibco) was used. Cell strain used was the human skin fibroblast (CCD-966Sk cell, Brand: ATCC®, CRL-1881), hereinafter referred to as CCD-966Sk cells.

The CCD-966Sk cells were seeded in a 6-well culture dish containing 2 ml of MEM culture medium per well at an amount of 1×10⁵ cells per well, cultured at 37° C. for 24 h, and divided into an experimental group, a comparison group and a control group. Then, the MEM culture medium was replaced with an experimental culture medium, and the experimental group and the comparison group were cultured at 37° C. for 24 h. The experimental culture medium in the experimental group was an MEM culture medium containing 0.25 mg/mL of Redlove apples extract prepared in Embodiment 1. The experimental culture medium in the control group was a pure MEM culture medium (namely the MEM culture medium without the Redlove apples extract).

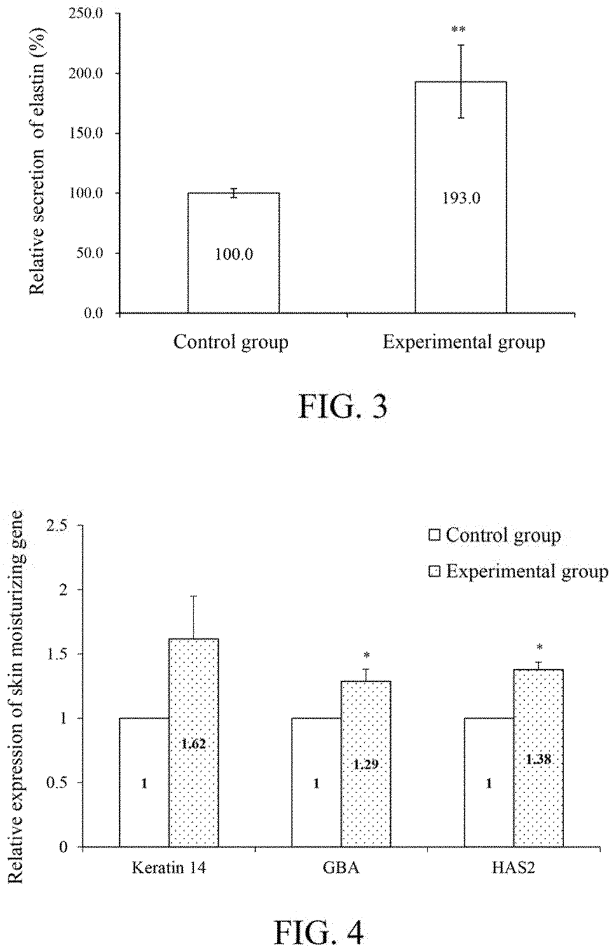

The CCD-966Sk cells of each group were treated by a Fastin™ Elastin Assay kit (Brand: Biocolor), and the elastin generation amount of the CCD-966Sk cells of two groups was detected by a full-spectrum optical analyzer (Brand: BioTek, Epoch), as shown in FIG. 3. The measured relative secretion of elastin of the control group without being cultured with the experimental culture medium for 24 h was regarded as 100%. Particularly, the statistical significant difference among the groups was statistically analyzed by a student's t-test. In FIG. 3, “**” represented that the p value was less than 0.01 under the comparison with the control group.

As shown in FIG. 3, compared with the control group, the relative secretion of elastin of the experimental group was 193.0%. In other words, the relative secretion of elastin of the experimental group was significantly improved by 1.93 fold(s) compared with the control group, which means that CCD-966Sk cells subjected to treatment of the Redlove apples extract for 24 h significantly secreted more elastin. Therefore, the Redlove apples extract had a function of promoting the cells to synthesize elastin, and further reducing skin collapse and wrinkles.

Therefore, the Redlove apples extract had a function of promoting the cells to synthesize elastin. After a subject taken the Redlove apples extract, skin cells of the subject were promoted to generate elastin, thus the skin elasticity was improved, skin collapse and wrinkles were reduced, and skin aging was delayed.

### Embodiment 5: Analysis Experiment of Keratinocytes Moisturizing Related Genes

Culture medium used was a special serum-free culture medium for keratinocytes (Keratinocyte-SFM (IX), purchased from Thermo, product number: 17005042) (hereinafter referred to as cell culture medium). Cell strain used was human primary epidermal keratinocytes HPEK-50 (Brand: CELLnTEC), hereinafter referred to as HPEK-50 cells. The analyzed keratinocytes moisturizing related genes were Keratin 14 (Krt14) gene (GeneID: 3861), Gluconoerrobesise (GBA) gene (GeneID: 2629) and Hyaluronan synthase 2 (HAS2) gene (GeneID: 3037).

The HPEK-50 cells were seeded in a 6-well culture dish containing 2 ml of cell culture medium per well at an amount of 1.5×10⁵ cells per well, cultured at 37° C. for 24 h, and divided into an experimental group and a control group. Then, the cell culture medium was replaced with an experimental culture medium, and cultured for 24 h. The experimental culture medium in the experimental group was a cell culture medium containing 0.125 mg/mL of Redlove apples extract prepared in Embodiment 1. The experimental culture medium in the control group was a pure culture medium (namely the cell culture medium without the Redlove apples extract).

Then, the RNAs of HPEK-50 cells cultured by the experimental culture medium of each group were extracted by an RNA extraction kit (Brand: Genemark). 1000 ng of extracted RNA was taken from each group to serve as a template, and the RNA from each group was translated into cDNA by a cDNA synthesis reagent (purchased from Geneaid Company, Taiwan) and SuperScript® III reverse transcriptase. Then, quantitative real-time reverse transcription polymerase chain reaction was performed by a KAPA CYBR FAST qPCR kit (KAPA Biosystems) and an ABI Step One Plus™ Real-Time PCR system in combination with a combined primer (shown in Table 2), so as to perform quantitative analysis on a target gene, and the analysis result was shown in FIG. 4. The instrument setting conditions of the quantitative real-time reverse transcription polymerase chain reaction were that: the reaction was performed at 95° C. for 23 s and performed at 60° C. for 30 s, and 40 cycles in total; and the cDNA PCR size of the Krt14 gene was 189 b, the cDNA PC R size of the GBA gene was 247 bp, and the cDNA PCR size of the HAS2 gene was 216 bp. Particularly, the gene expression in FIG. 4 was relatively presented, the standard deviation was calculated by an STDEV formula of Excel software, and the statistical significant difference among the groups was statistically analyzed by a student's t-test.

TABLE 2

| Sequence | Sequence | | |
|---|---|---|---|
| number | name | Sequence (5′→3′) | Length |
| SEQ ID NO: 3 | Krt14-F | TTCTGAACGAGATGCGTGAC | 20 |
| SEQ ID NO: 4 | Krt14-R | GCAGCTCAATCTCCAGGTTC | 20 |
| SEQ ID NO: 5 | GBA-F | TCCAGTTGCACAACTTCAGC | 20 |
| SEQ ID NO: 6 | GBA-R | TTGTGCTCAGCATAGGCATC | 20 |
| SEQ ID NO: 7 | HAS2-F | AAGAACAACTTCCACGAAAAGGG | 23 |
| SEQ ID NO: 8 | HAS2-R | GGCTGGGTCAAGCATAGTGT | 20 |

As shown in FIG. 4, the expression level of each gene in the control group was regarded as 1, which means that the expression level of the keratinocyte moisturizing related genes in the HPEK-50 cells without being subjected to treatment of the Redlove apples extract was regarded as 100%. Therefore, the expression level of the Keratin 14 gene in the experimental group was 1.62, the expression level of the GBA gene was 1.29, and the expression level of the HAS2 gene was 1.38. In other words, the expression level of the Keratin 14 gene in HPEK-50 cells subjected to treatment of the Redlove apples extract was improved by 1.62 fold(s), the expression level of the GBA gene was improved by 1.29, and the expression level of the HAS2 gene was improved by 1.38 fold(s).

Therefore, the Redlove apples extract had an effect of improving the expression level of the Keratin 14 gene, the GOA gene and the HAS2 gene. In other words, after a subject taken the Redlove apples extract, the expression level of the Keratin 14 gene, the GBA gene and the HAS2 gene in the subject were improved, thus the skin structure was enhanced, the cell gaps were reduced, the optimal moisturizing factor was provided for the skin, and the skin was softer and well-moisturized.

### Embodiment 6: Human Trial

The skin condition changes of 8 subjects before and after taking a composition including the Redlove apples extract were compared by a self-control mode. The used composition was a 50 mL bottle of Redlove apples drink, and each bottle of Redlove apples drink contained 0.6 g of Redlove apples extract.

Test mode: each of the 8 subjects taken a bottle of Redlove apples drink every day, continuously taken the drink for 4 weeks, and detected the skin wrinkles, textures and the whole skin conditions before taking (the 0^{th} week) and 4 weeks after taking (the 4^{th} week) the Redlove apples drink. A used detection instrument was a full-face detector (VISIA Complexion Analysis System (Canfield scientific, USA)).

Wrinkle detection: a high-resolution skin image was shot with visible light (white light) of the VISIA Complexion Analysis System (Canfield scientific, USA), and analysis and calculation were carried out by built-in software according to the length and depth of wrinkles. The higher the detected value was, the more serious the wrinkle condition of the subject was.

Skin texture detection: a high-resolution skin image was shot with visible light (white light) of the VISIA Complexion Analysis System (Canfield scientific, USA), and coarseness analysis was carried out by built-in software according to the depression and bulge of the skin. The higher the detected value was, the coarser the skin of the subject was.

Particularly, the statistical significant difference among the measurement results of the 0^{th} week and the 4^{th} week was statistically analyzed by the student's t-test.

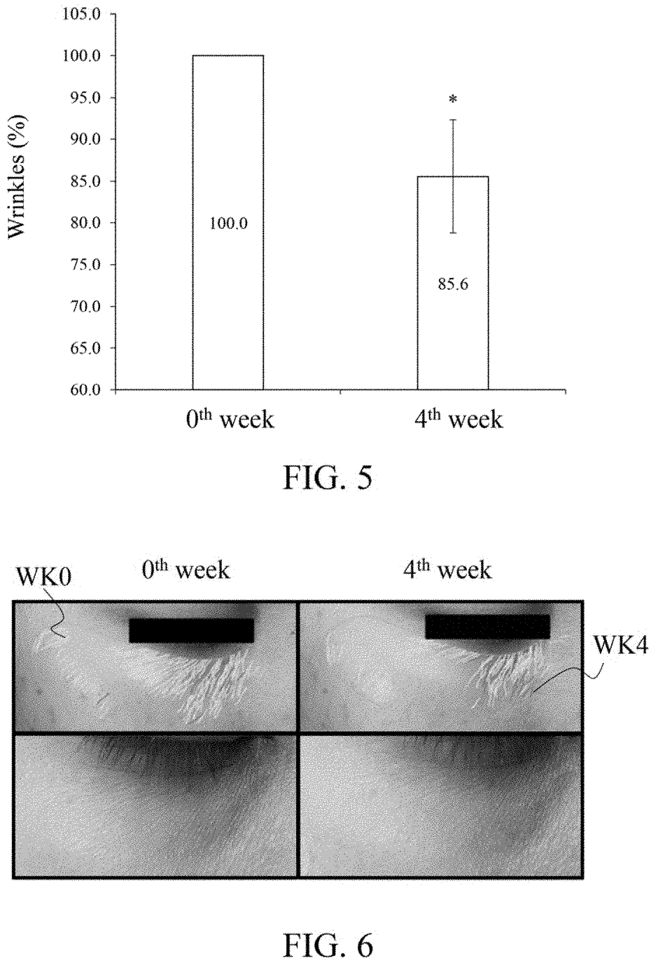

In the aspect of reducing wrinkles, 6 subjects in the 8 subjects felt the wrinkle reduction, representing that 75% of the persons feel the improvement. As shown in FIG. 5. An average value of the wrinkles, measured at the 0^{th} week, of the 8 subjects was regarded as 100%. Compared with the average value of the wrinkles measured at the 0^{th} week, an average value of the wrinkles measured at the 4^{th} week was 85.6%. In other words, after the subjects taken the Redlove apples drink containing the Redlove apples extract for 4 consecutive weeks, the wrinkles of the 8 subjects were averagely reduced by 14.4%. As shown in FIG. 6, one of the 8 subjects had many under-eye wrinkles WK0 during the measurement at the 0^{th} week, and the under-eye wrinkles WK4 were reduced after the subject taken the Redlove apples drink for 4 consecutive weeks. Therefore, after the subjects taken the composition including the Redlove apples extract, the wrinkles were reduced, and the fine lines were effectively smoothed.

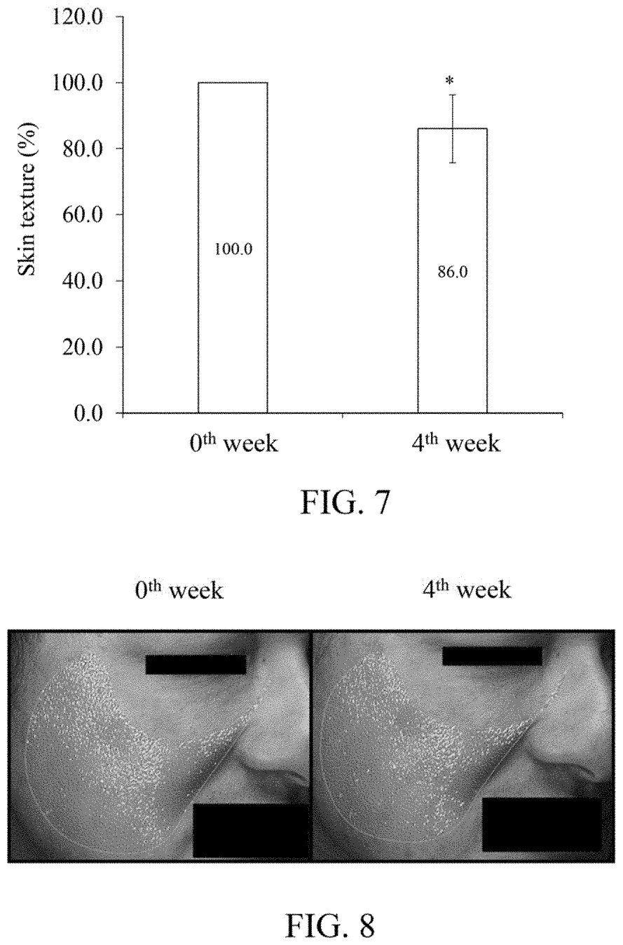

In the aspect of improving skin texture, 7 subjects in the 8 subjects felt the skin texture reduction, representing that 87.5% of the persons feel the improvement. As shown in FIG. 7. An average value of the skin textures, measured at the 0^{th} week, of the 8 subjects was regarded as 100%. Compared with the average value of the skin textures measured at the 0^{th} week, an average value of the skin textures measured at the 4^{th} week was 86.0%. In other words, after the subjects taken the Redlove apples drink including the Redlove apples extract for 4 consecutive weeks, the wrinkles of the 8 subjects were averagely reduced by 14%. As shown in FIG. 8, one of the 8 subjects had many densely-distributed skin textures during the measurement at the 0^{th} week, and the skin textures was reduced after the subject taken the Redlove apples drink for 4 consecutive weeks. Therefore, after taking the composition including the Redlove apples extract, the coarseness of the skin could be reduced.

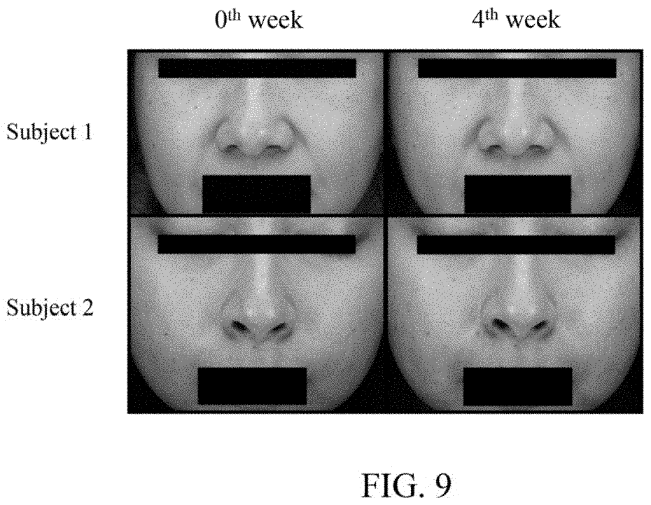

As shown in FIG. 9, two subjects in the 8 subjects was detected the skin conditions with the full-face detector at the 0^{th} week and the 4^{th} week respectively. After the subjects taken the Redlove apples drink for 4 consecutive weeks, the skin conditions of the two subjects became ruddy and glossy. Therefore, after taking the composition including the Redlove apples extract, the skin condition were effectively improved.

In conclusion, the Redlove apples extract of any one of the embodiments of the instant disclosure can be used for improving the skin condition (such as reducing wrinkles, reducing skin textures, making the skin ruddy and glossy, or a combination thereof). The Redlove apples extract is prepared by extracting the Redlove apples (Malus pumila) with water at 95° C. for 1 h. In some embodiments, the Redlove apples extract has the functions of increasing the expression level of the elastin synthesis related gene (such as FBN1 gene), increasing the skin elastin content, increasing the expression level of the skin moisturizing related genes (such as Keratin 14 gene, GBA gene and HAS gene), and the like, so that the skin condition is effectively improved (such as reducing fine lines, reducing skin coarseness, making the skin fine, and making the skin ruddy and glossy).

Although the instant disclosure has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A method for improving skin condition with a Redlove apples extract, comprising: administering to a subject in need thereof a composition comprising an effective dose of the Redlove apples extract, wherein the Redlove apples extract is prepared by extracting Redlove apples (Malus pumila) with water at 80° C. to 90° C. for 45 min to 75 min.

2. The method according to claim 1, wherein the Redlove apples extract is prepared by extracting the Redlove apples with water at 85° C. for 60 min.

3. The method according to claim 1, wherein improving the skin condition comprises reducing wrinkles, reducing skin textures, making the skin ruddy and glossy, or a combination thereof.

4. The method according to claim 1, wherein the Redlove apples extract contains at least 129.1 ppm of chlorogenic acid.

5. The method according to claim 1, wherein the Redlove apples extract has a capability of increasing an expression level of elastin synthesis related gene.

6. The method according to claim 5, wherein the elastin synthesis related gene comprises FBN1 gene.

7. The method according to claim 1, wherein the Redlove apples extract has a capability of increasing skin elastin content.

8. The method according to claim 1, wherein the Redlove apples extract has a capability of increasing an expression level of skin moisturizing related genes.

9. The method according to claim 8, wherein the skin moisturizing related genes comprise Keratin 14 gene, GBA gene and HAS gene.

10. The method according to claim 1, wherein the effective dose of the Redlove apples extract in liquid form is 0.6 g/day.
